(19) United States
(12) Patent Application Publication
(10) Pub. No.: US 2018/0289324 A1
(43) Pub. Date: Oct. 11, 2018

(54) AUTOMATIC ASSESSMENT OF THE SQUAT QUALITY AND RISK OF KNEE INJURY IN THE SINGLE LEG SQUAT

(71) Applicants: Rezvan Kianifar, Waterloo (CA); Dana Kulic, Waterloo (CA)

(72) Inventors: Rezvan Kianifar, Waterloo (CA); Dana Kulic, Waterloo (CA)

(21) Appl. No.: 15/826,259

(22) Filed: Nov. 29, 2017

Related U.S. Application Data
(60) Provisional application No. 62/531,660, filed on Jul. 12, 2017.
(60) Provisional application No. 62/427,529, filed on Nov. 29, 2016.

(51) Int. Cl. A61B 5/00; A61B 5/11

(52) U.S. Cl. CPC A61B 5/4884; A61B 5/1121; A61B 5/4585; A61B 2562/0223; A61B 5/1124; A61B 2503/10; A61B 2562/0219; A61B 5/7275

(57) ABSTRACT

Provided herein is a method for automatic assessment of squat quality using a quantitative measure of a mobility test, such as a single leg squat (SLS) performance. The method is useful in clinical assessment protocols for rehabilitation, sports medicine, and orthopedic knee surgery assessment.

### RELATED APPLICATION

This application claims priority to U.S. Provisional Patent Application No. 62/531,660, filed on Jul. 12, 2017 and U.S. Provisional Patent Application No. 62/427,529, filed on Nov. 29, 2016, each of which is incorporated herein by reference in its entirety.

### BACKGROUND

Many clinical assessment protocols rely on mobility tests, where the patient is asked to perform a target movement. The clinician observes the movement performance of the patient and generates an assessment. The single leg squat (SLS) is a mobility test commonly used in rehabilitation, sports medicine and orthopedic knee surgery assessment [1], [26]. Correct performance of the SLS is a key factor for diagnosis and assessment of recovery. The SLS rating is based on the degree of inward movement of the knee, known as medial knee displacement or dynamic knee valgus (DKV).

DKV correlates with non-contact Anterior Cruciate Ligament (ACL) injury and patellofemoral pain [2]. The SLS test helps with early screening of those at higher risk of ACL rupture, which happens frequently among athletes involved in high risk sports such as soccer, football, basketball, and lacrosse [3].

More than 120,000 ACL injuries occur annually, most during the high school years [3]. Treatment in 90% of patients includes reconstruction surgery, followed by a rehabilitation period [4]. The estimated average annual treatment cost of ACL rupture in the United States is more than 2 billion dollars [5]. Return to play for professional athletes following ACL surgery can take almost one year [6]. More than 50% will not return to their pre-injury level of performance [4] and between 50% to 100% develop osteoarthritis within 5 to 10 years after surgery. Moreover, the risk of second injury increases up to 5 times in those who have undergone initial surgery as compared to those without an initial surgery [4]. All these statistics highlight the importance of early screening of individuals at higher risk, through mobility tests such as the SLS or the drop jump.

Current SLS assessment is based on visual observation by the clinician. Therefore, diagnosis is subjective and depends on the experience of the clinician. In addition, since therapists see a large number of patients each day, it may be difficult for them to remember the previous condition of each patient without a quantitative history for each person. Finally, physical therapists (PTs) cannot ensure that the patient has done all the prescribed exercises at home, so they rely on patients' self-reports. Using an automated assessment method, better diagnosis and treatment may be possible. Furthermore, the assessment method combined with a feedback protocol can be applied at home, in the absence of a PT, which ensures correct performance of the prescribed exercises and faster rehabilitation.

An automated assessment method can help with early detection of DKV among young athletes and with identifying those at higher risk of injury, and also help to assist orthopedic and rehabilitation professionals with patient assessment and to provide a record of past performance, leading to better treatment protocols.

SLS and other mobility tests such as the double leg squat and double leg jump have been widely investigated in clinical and sport medicine studies. The main purpose of the majority of these studies is to find relationships between the occurrence of knee valgus during the mobility test and factors such as age, gender, body mass index, history of injury, and kinematic or neuromuscular characteristics of the subjects (usually athletes) [7], [8], [9], [2], [10], [11].

Finding these predictors helps in coming up with appropriate preventive strategies. For example, if it is found that hip adductor muscle weakness correlates with poor performance (DKV occurrence) in SLS, then specific exercises can be prescribed to improve that muscle.

Zeller et al. [8] investigated the difference between kinematics and muscular activity of 9 men and 9 women athletes during SLS. Kinematic parameters including 3 dimensional trunk, hip, knee, and ankle joint angles were obtained from marker-based motion analysis. Electromyographic activity was measured via surface electrodes. The collected data was analyzed using one-way analysis of variance. According to their results, women exhibited more knee valgus, which was associated with greater ankle dorsiflexion and pronation, less trunk lateral flexion, and greater hip adduction (Add.), flexion (Flex.), and rotation. Rectus femoris muscle activation was also greater in women.

Bittencourt et al. [9] investigated hip and foot contributions to high dynamic knee valgus during SLS and at the landing moment of a double leg jump among 173 athletes. Data were collected in a motion capture studio and the frontal plane knee projection angle was measured at 60° of knee flexion and during a static single-leg stance. Four other measures, including the passive range of motion (ROM) of the hip internal rotation (IR), the isometric strength of the dominant-limb hip abductors, the shank-forefoot alignment and participants' sex were defined as features to be input into a classification and regression Tree. Their results indicated that high dynamic knee valgus can be predicted by decreased hip abductor torque and increased passive ROM of the hip IR for both the SLS and double leg jump landing.

Padua et al. [2] compared the neuromuscular characteristics of a group of 18 individuals with excessive knee valgus with a control group of 19 healthy individuals during double leg squat performance. Electromyography (EMG) was used for muscle activation measurement. Individuals were assigned to either the control or DKV group based on an evaluation by an expert rater. A correlation between increased hip-adductor activation and increased coactivation of the gastrocnemius and tibialis anterior muscles was reported.

In a similar study, Stiffler et al. [10] compared kinematic characteristics including ROM and postural alignment of 97 healthy individuals during the double leg jump, in order to find differences between those with and without excessive DKV. Motion labeling was based on total Landing Error Scoring System (LESS) [12]. Their results showed associations between DKV and less ankle dorsiflexion, as well as higher Q angle.

Ugalde et al. [11] investigated the relationship between the occurrence of DKV and age, gender, and body mass index of the 142 middle and high school athletes both in SLS and the drop jump test. Their results showed significantly lower knee-hip ratio for individuals with DKV during SLS. However, they found no relationship between DKV and age, gender, or body mass index.

The focus of the all of the above studies was identifying correlates of DKV. Generally, these studies first detected positive DKV occurrence based on expert clinician observations or manual measurements extracted from video frames. Very few studies have tried to develop an automatic algorithm for DKV detection,

Whelan et al. [13] classified SLS repetitions of 19 healthy participants into correct and incorrect using a single lumbar-mounted IMU. The investigators extracted time domain features from accelerometer and gyroscope measurements, the IMU orientation (represented as roll, pitch, yaw), and accelerometer magnitude. Using the generated feature vector and labels provided by an expert, they trained a Random Forest classifier, which achieved 92.1% accuracy with repeated random-sample validation. Despite these promising results, these data were not clinically interpretable, as features are defined based on direct acceleration and gyroscope output signals. Furthermore, the Leave One Subject Out cross validation (LOSO-CV) result was not reported while in clinical applications, as previously unseen subjects have to be analyzed.

There remains a need in the art for an automatic assessment system for the SLS test.

### SUMMARY

Provided herein is a method of assessing the risk of knee injury in a subject, comprising determining a performance parameter of the squatting leg of the subject performing a leg squat, wherein the parameter is measured by means of one or more sensor(s) placed on the subject, and analyzing the parameter to obtain an indication of the risk of knee injury in the subject.

In an embodiment, the leg squat is a single leg squat (SLS).

In an embodiment, the sensor is an inertial measurement unit (IMU) composed of a set of three accelerometers measuring linear accelerations, and three gyroscopes measuring angular velocities and a magnetometer measuring earth's magnetic field.

In an embodiment, several parameters are measured by the sensor including joint angle, velocity and acceleration of the squatting leg.

In an embodiment, a set of three IMU's are employed.

In an embodiment, the IMU's are located individually at the lower back, at the thigh and at the tibia of the subject.

In an embodiment, dynamic knee valgus (DKV) is assessed by analysis of the parameters and DKV is used as an indication of the risk of the knee injury in the subject.

In an embodiment, readout of the sensor is received remotely.

In an embodiment, the analysis includes evaluation of flexion at the hip and knee and hip and ankle rotation.

### BRIEF DESCRIPTION OF THE DRAWINGS

FIG. 1 is a set of photographs showing Single Leg Squat Performance. Left: Good SLS performance. Right: Inward movement of the knee during poor SLS called Dynamic Knee Valgus (DKV).

FIG. 2 is a depiction of a 7 DOF Kinematic Model (Pilot Study) and a set of photographs. Shown are a 7 DOF kinematic model of the left leg (left) and photographs of sensor placement (right). The kinematic link lengths were either measured or obtained from the motion capture marker information.

FIG. 3 is a depiction of a Decision Tree Classifier. Shown is a Decision Tree structure for LOSO cross validation for the 3 class (right) and 2 class (left) problems, where x1 corresponds to ankle IR ROM and x2 corresponds to MAD of ankle IR angle. For the 2-class problem, poor squats are detected when ankle IR ROM (x1) is greater than 0.38 rad (20.630). For the 3-class problem, MAD (x2) of ankle IR angle greater than 0.26 rad (14.90) identifies good squats. MAD of ankle IR angle less than 14.9° indicates either moderate or poor squats, which are again differentiated based on ankle IR ROM (x1).

FIG. 4 is a drawing of a 7 DOF Kinematic Model Shown is a 7 DOF kinematic model of the left leg including the 3 DOF ankle joint, 1 DOF knee joint, and 3 DOF hip joint.

FIG. 5 is a graph of a Segmentation of Joint Angle Trajectory. Segment points (green arrows) were found by detecting peaks (red arrows) of low pass filtered knee joint angle and computed the midpoint of the peak to peak distances (horizontal arrows).

FIG. 6 shows a series of graphs of segmented joint angles. The data are shown without low pass filtering used for feature extraction.

### DETAILED DESCRIPTION

The following description of the invention is merely intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

Unless the context requires otherwise, throughout the specification and claims, the word “comprise” and variations thereof, such as “comprises” and “comprising” are to be construed in an open, inclusive sense, that is as “including, but not limited to”.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Provided herein is a method of assessing the risk of knee injury in a subject, comprising determining a performance parameter of the squatting leg of the subject performing a leg squat, wherein the parameter is measured by means of one or more sensor(s) placed on the subject, and analyzing the parameter to obtain an indication of the risk of knee injury in the subject.

The term “squat”, as used herein, is a compound exercise that trains primarily the muscles of the thighs, hips and buttocks, quadriceps femoris muscle, hamstrings, as well as strengthening of the bones, ligaments and insertion of the tendons throughout the lower body.

The movement begins from a standing position and is initiated by moving the hips back and bending the knees and hips to lower the torso, then returning to the upright position. In an embodiment, the leg squat is a single leg squat (or SLS), where the movement begins and ends while the subject is on one leg.

The term “sensor”, as used herein, is a device, module, or subsystem that detects events or changes in its environment and sends the information to a device or other electronics, such as a computer or computer processor. In an embodiment, readout of the sensor is received remotely.

The sensor may be attached either to the body or to an article of clothing worn by the subject. In an embodiment, the sensor is an inertial measurement unit (IMU) composed of a set of three accelerometers measuring linear accelerations, and three gyroscopes measuring angular velocities and a magnetometer measuring earth's magnetic field. An IMU useful for the invention may measure and report certain parameters, including, but not limited to, the body's specific force, angular rate, and magnetic field surrounding the body. In an exemplary embodiment, the parameters measured include joint angle, velocity and acceleration of the squatting leg.

The term “dynamic knee valgus” or “DKV” refers to an abnormal movement pattern of the lower extremity, characterized by hip adduction and hip internal rotation, typically when in a hips-flexed position.

Provided herein is a method of assessing the risk of knee injury in a subject, comprising determining a performance parameter of the squatting leg of the subject performing a leg squat, wherein the parameter is measured by means of one or more sensor(s) placed on the subject, and analyzing the parameter to obtain an indication of the risk of knee injury in the subject.

In an embodiment, the leg squat is a single leg squat.

In an embodiment, the sensor is an inertial measurement unit composed of one or more accelerometers. In various embodiments, the sensor is an inertial measurement unit composed of a set of three accelerometers measuring linear accelerations, and three gyroscopes measuring angular velocities and a magnetometer measuring earth's magnetic field.

In an embodiment, at least one parameter is measured by the sensor. For example, the parameter is joint angle, velocity and acceleration of the squatting leg.

In an embodiment, a set of one or more IMUs are employed. For example, three IMU's are employed. In an embodiment, the IMUs are located at different locations on the body. For example, the IMUs are individually placed. In various embodiments, the IMUs are placed at the lower back, at the thigh and at the tibia of the subject.

In an embodiment, DKV is assessed by analysis of at least one parameter. In an embodiment, the DKV is used as an indication of the risk of the knee injury in the subject.

In an embodiment, readout of the sensor is received remotely. In an embodiment, readout of the sensor is displayed on a device. For example, the device comprises a screen, display or monitor.

In an embodiment, the analysis includes evaluation of flexion. For example, the analysis includes evaluation of flexion at a location in the body. For example, the analysis includes evaluation of flexion at the hip. In an embodiment, the analysis includes evaluation of flexion at the knee. In an embodiment, the analysis includes evaluation of flexion ankle rotation.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means without the exercise of inventive capacity and without departing from the scope of the invention.

### EXAMPLES

### Example 1: Pilot Study—Design

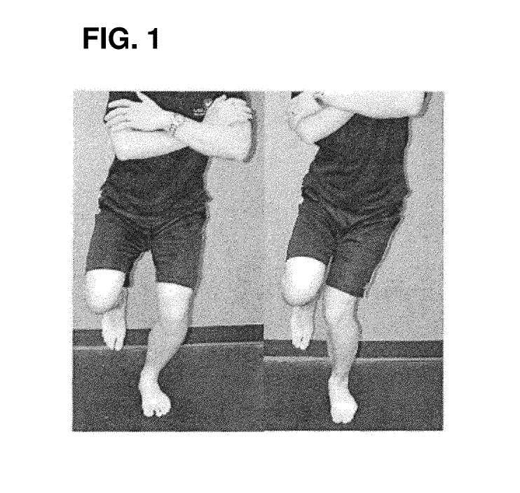

An approach for automated SLS classification based on joint kinematics was proposed. First, an Extended Kalman Filter based method [15] was used to estimate ankle, knee, and hip kinematic parameters during SLS from IMU measurements. See FIG. 1. Time domain features were then extracted from these measurements; the most informative ones were selected via feature selection. Based on an expert labeled dataset, classifiers were then trained to distinguish between good, poor and moderate squats.

### A. Pose Estimation

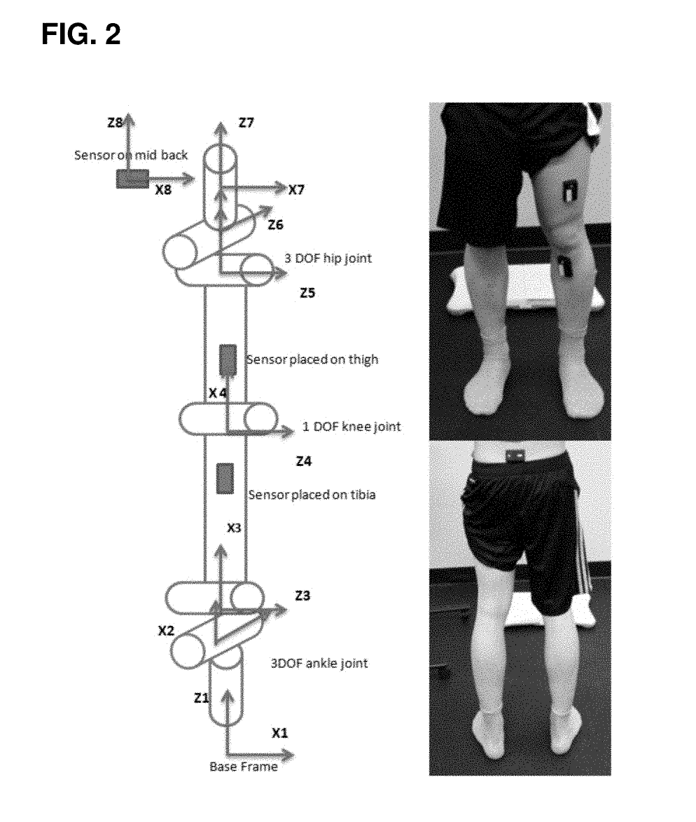

To develop an automated DKV assessment system suitable for clinical use, it is preferable to measure joint angles, as they best describe the occurrence of DKV in clinically interpretable terms. For this purpose, the pose estimation algorithm proposed in [15] was adapted to estimate the joint angles, velocities and accelerations during SLS using IMUs. A kinematic model of the lower leg, consisting of a 3 Degree of Freedom (DOF) ankle joint, 1 DOF knee joint, and 3 DOF hip joint (as depicted in FIG. 2) (left) and the IMU measurements were fused via an Extended Kalman Filter to recover the joint angle, velocity, and acceleration of each DOF. See also [15].

### B. Feature Generation

Various feature extraction methods have been used for human activity recognition [18]. The mean, standard deviation (STD), variance (VAR), interquartile range (IQR), mean absolute deviation (MAD), correlation between axes, entropy, and kurtosis are among the time domain features commonly used for activity recognition from the acceleration signal [18]. In a similar review, Preece et al. [27] have identified the mean, median, variance, skewness, kurtosis and interquartile range as commonly used time domain features.

In this study, all of the commonly used features were generated and feature selection techniques were used to identify the best features from the data. The features generated in this study include: the mean, root mean square (RMS), STD, VAR, MAD, skewness, kurtosis, range, minimum, and maximum of the joint angle, velocity and acceleration of each DOF during each repetition of a SLS.

### C. Feature Selection

The purpose of feature selection in this study was not only to reduce the dimensionality, but also to identify which factors are best predictors of DKV. Due to the importance of feature selection, 18 different feature selection techniques were tried and those identified by the majority of the methods were chosen as the selected features. To identify the majority, features which were among the top 10 ranked by each algorithm and repeated more than 8 times (selected by at least half of the methods as the top ten) were reported as the best predictors of DKV. In addition to the feature selection methods, Supervised Principal Component Analysis (SPCA) was also applied to the data for comparison.

For feature selection, available MATLAB packages from Arizona State University [19] and from Pohjalainen et al. [20] were used. Pohjalainen's package included five different techniques: Mutual Information, Statistical Dependency, Random Subset Feature Selection, Sequential Forward Selection, and Sequential Floating Forward Selection. The ASU package included 12 techniques: Correlation based Feature Selection, ChiSqaure, Fast Correlation-Based Filter, Fisher Score, Gini Index, Information Gain, Kruskal-Wallis, Minimum-Redundancy-Maximum-Relevance selection, Relief-Feature selection strategy, Sparse Multinomial Logistic Regression via Bayesian L1 Regularization, T-test, and the Bayesian logistic regression. Least Absolute Shrinkage and Selection Operator (LASSO) was also implemented using MATLAB's default function. For SPCA, the MATLAB code developed by Barshan et al. [21] was utilized.

### D. Classification

Three different classifiers, the Support Vector Machine, Linear Multinomial Logistic Regression, and Decision Tree, were tried for both the 2 class and 3 class classification problems. All classifiers were implemented using MATLAB R2014b. SVM was selected as it is robust to small training data size. For 3 class classification, one-versus-all and one-versus-one SVM with linear kernel were implemented. SVM multi-label results were computed by majority vote between one-vs-one classification results. The Decision Tree is beneficial as it provides threshold values (cutoff points) in the selected features which can be informative for clinical interpretation.

### Example 2. Pilot Study—Classification of Squat Quality with Intertial Measurement Units in the Single Leg Squat Mobility Test

### I. Experiments

Seven participants (6 male and 1 female; mean age 32.3±11.6 years) took part in this study. Inclusion criteria were adults not having any lower back or leg injuries in the past six months. The experiment was approved by the University of Waterloo Research Ethics Board, and all participants signed a consent form prior to the start of data collection.

### A. Data Collection

Three Yost [22] IMU sensors were affixed to the participant using hypoallergenic tape. Sensor placement sites included the low back at the level of the first sacral vertebra, the anterior thigh 10 cm above the patella aligned with the sagittal plane, and the lower leg on the flat surface of the tibia at the level of the tibial tubercle, as illustrated in FIG. 2 (right). Due to wireless communication, sampling rates were not consistent or identical for all sensors. The average sampling rate was 90±10 Hz. All sensors were interpolated and resampled to the same rate (100 HZ). Participants were instructed to remove their shoes and socks, and stand on their dominant leg (the leg they would kick a ball with) with toes pointing straight ahead, while keeping their weight centered over the ball of the foot and their arms crossed in front of their body. In each trial, participants performed five consecutive cycles of the SLS movement. For the SLS collection to be deemed successful, the subject had to perform the squat without allowing the legs to contact each other, and without losing balance (i.e., without having the non-weight bearing leg touch the ground).

### B. Data Labeling

Three of the participants replicated good, poor, and moderate squats under the instruction and supervision of an expert clinician; the other participants performed the squats naturally. The naturally performed squats were labelled by an experienced movement scientist using a modified qualitative SLS clinical rating tool [28]. A SLS was rated “good” if DKV did not occur during the squat or if DKV occurred, the patella did not have a trajectory that pointed towards the second toe; “moderate” if the patella pointed toward or past the second toe, but did not point past the inside aspect of the foot; and “poor” if the patella pointed past the inside aspect of the foot. To ensure a balanced dataset, we made use of all the natural squats (which were mostly bad or moderate) and supplemented with the replicated exemplars.

The number of trials was not the same for all participants. There were 7 labeled trials available from participant 2 (3 good, 1 moderate and 3 poor), 6 from participant 1 and participant 3 (1 good, 1 poor, and 1 moderate for each), 1 from participant 4 (poor), 2 from participant 7 (moderate), 3 from participant 5 (2 poor and 1 good) and 1 from participant 6 (moderate). Each trial consisted of 5 consecutive squats, which resulted in 100 examples of SLS including 30 examples of good, 30 examples of moderate, and 40 examples of poor squats.

Given the 7 DOF kinematic model, where each DOF includes an estimate of its position, velocity, and acceleration, the total number of features for each segment or observation was 210. Therefore, the final data set had 100×210 dimensions. Another dataset was also produced with the same features, but including only good and poor data (i.e., excluding the moderate SLS data) which had 70 observations. All data were normalized to bring values in [01] range. Zero velocity crossing criteria [29] were used to segment continuous time series data into five squats.

### II. Results and Discussion

The feature selection results are summarized in Table A. The feature selection results highlight the importance of the ankle IR angle features for differentiating good, moderate and poor squats. Although according to clinical studies [9], [8], the hip plays an important role in DKV, the feature selection results in this study suggest that good classification can be performed based on only the ankle kinematics.

TABLE A
FEATURES RANKED AS TOP TEN BY MORE THAN 8 FEATURE
SELECTION TECHNIQUES.

| Selected features For 2 | | Selected features For 3 | |
|---|---|---|---|
| class problem | N_r | class problem | N_r |
| ROM of ankle IR | 14 | STD of ankle IR angle | 13 |
| STD of ankle IR angle | 11 | VAR of ankle IR angle | 13 |
| MAD of ankle IR angle | 11 | MAD of ankle IR angle | 13 |
| VAR of ankle IR angle | 10 | ROM of ankle IR | 12 |
| RMS of ankle IR vel. | 9 | | |
| MAD of ankle IR vel. | 9 | | |
| RMS of ankle adduc. acc. | 9 | | |

N_r: Number of times ranked as top ten features

Classification results for 2 class and 3 class problems are reported in Table B for both 10 fold CV and LOSO cross validations. For reporting the accuracy, the number of selected features or Principal Components (PCs) in SPCA was set to one first and accuracy was calculated. Then, the number of features or PCs was increased one by one up to the point that further increases did not improve performance. The reported accuracies are the best performance each classifier achieved. Matrix inversion with the full dimensional dataset was not possible with LMLR; therefore no results are reported for this condition.

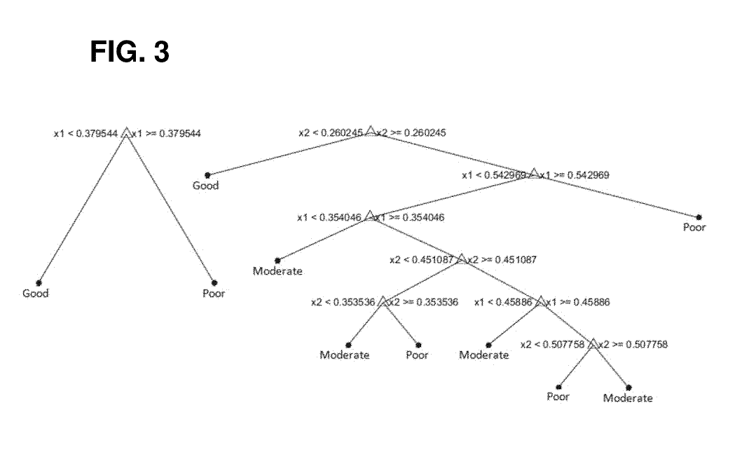

Analysis of the decision tree results using majority selected features shows that for both LOSO and 10 fold CV, the best performance was achieved using only the ankle IR ROM feature for the 2 class problem, while for the three class problem, ROM and MAD of the ankle IR angle resulted in best accuracy for LOSO CV and STD and MAD of the ankle IR angle for 10 fold CV. The decision tree structure for the 2 and 3 class problems is shown in FIG. 3.

The classification results in Table B show very good accuracy for almost all of the classifiers in the 2 class classification problem, which suggests that differentiation between good and poor squats is achievable. For 10 fold CV, the best performance was obtained with SVM using the full dimensional data. SPCA resulted in the best performance for all three classifiers in LOSO CV, indicating that the 10-fold CV results using all the features may be overfitted. With regard to dimensionality reduction, SPCA in combination with all three classifiers resulted in better accuracy than subset selection methods; however, features extracted by SPCA are difficult to interpret clinically. For the three class problem, again SVM using the full dimensional data outperformed other classifiers in 10 fold CV. However, for the LOSO cross validation, the combination of Decision Tree and SPCA (first four PCs) resulted in the best accuracy. As expected, classification of the moderate squat is most difficult, showing the lowest accuracy in the one-vs-all and moderate-vs-poor SVM results.

TABLE B
ACCURACIES (%) FOR THE 2-CLASS AND 3-CLASS CLASSIFICATION PROBLEMS
USING THREE CLASSIFIERS AND TWO DIFFERENT CROSS-VALIDATION METHODS

| | | Validation method | | | | | |
|---|---|---|---|---|---|---|---|
| | | 10 Fold CV | | | LOSO CV | | |
| | | Majority | | | Majority | | |
| # of | Dimensionality | Selected | | No | Selected | | No |
| Classes | reduction method | features | SPCA | reduction | features | SPCA | reduction |
| 2 class | SVM | 95.7143 | 98.5714 | 99.7143 | 88.5714 | 98.5714 | 75.7143 |
| | Logistic Regression | 93.5714 | 98.5714 | — | 91.4286 | 98.5714 | — |
| | Decision Tree | 92.4286 | 98.5714 | 95.8571 | 87.1429 | 98.5714 | 81.4286 |
| 3 class | SVM good vs all | 91.8 | 84.7 | 98.1 | 91 | 83 | 79 |
| | SVM poor vs all | 77.8 | 87.4 | 96.9 | 75 | 82 | 72 |
| | SVM moderate vs all | 64.8 | 77.5 | 87.2 | 62 | 74 | 42 |
| | SVM good vs moderate | 91.6667 | 100 | 99 | 91.6667 | 73.3333 | 62.3333 |
| | SVM poor vs moderate | 70.1429 | 90 | 96.7143 | 67.1429 | 80 | 50 |
| | SVM good vs poor | 93.7143 | 97.5714 | 99.4286 | 91.4286 | 97.1429 | 75.7143 |
| | SVM majority vote | 74.2 | 93.2 | 96.6 | 72 | 70 | 46.4 |
| | Logistic Regression | 73.6 | 93.1 | — | 68 | 68 | — |
| | Decision Tree | 70.2 | 83.5 | 77.6 | 62 | 73 | 68 |

### III. Discussion and Conclusions

For the dataset in this study, good and poor squats of an unseen subject were classified with 98.6% accuracy using SVM and SPCA for dimensionality reduction. In the 3 class case, 73% accuracy was achieved with a decision tree and SVM. There was no significant difference in classification performance between subjects who performed natural squats versus those who replicated good, poor and moderate squats, suggesting that replicated movements were similar to natural movements. Feature selection results emphasized the ankle internal rotation joint angle features for determining squats quality, suggesting that it may be possible to achieve good classification of the SLS by using only a simple 3 DOF model to estimate ankle joint kinematics. This is advantageous, as it simplifies the pose estimation and reduces the number of sensors from 3 to 1, reducing the complexity of the measurement apparatus and the setup and computation procedure. Similar clinical studies [9] used time consuming manual measurements and focused on only the feature selection part, while the method in this study is completely automated and simple to apply, and therefore more easy to apply in the clinical setting.

### Example 3: Large Study—Design

The purpose of this study was to develop an automatic assessment system for the SLS test. An IMU-based method is used for joint angle, velocity and acceleration estimation of the squatting leg. Statistical and time domain features are generated from these measurements. The most informative features are selected using a combination of different feature selection techniques and used as input for supervised classifier training. A dataset of SLS performed by healthy participants was collected and labeled by three expert raters. The raters applied two different labeling criteria including the observed amount of DKV during the motion and evaluated risk of injury for the participant based on SLS performance. The expert raters rated each SLS repetition quality as poor, moderate or good and she risk of injury as high, mild, or none. The labeled data was used to train classifiers for each assessment criterion. The results showed excellent discrimination between good and poor SLS, and also between high risk and low risk participants.

In the current study, a similar approach to the pilot study was applied to a larger dataset including a similar number of male and female participants, whose performances are labeled by clinical experts using two different criteria: amount of knee valgus and risk of knee injury. Additional data analysis was also performed based on gender specific datasets and ankle only features.

In order to find clinically interpretable predictors of knee valgus and risk of injury, an EKF-based pose estimation method [15] was first applied to extract lower body joint parameters. The time series data of the estimated joint angles were then segmented into single squats, from which statistical time domain features were extracted and used for feature selection and classification.

### A. Pose Estimation

For automated SLS assessment, a set of three IMUs were employed to track lower body motion during the squats. An IMU is a compact package composed of an accelerometer measuring linear acceleration, angular velocity and a magnetometer measuring the earth's magnetic field. The magnetometer is not usually used in pose estimation, as it is subject to interference by ferromagnetic objects [16].

Clinical rating of the SLS includes the visual assessment of kinematic joint parameters, especially the joint angles. Therefore, to provide a clinically interpretable assessment method, instead of using raw IMU outputs, joint angles, velocities and accelerations were extracted and used for classification.

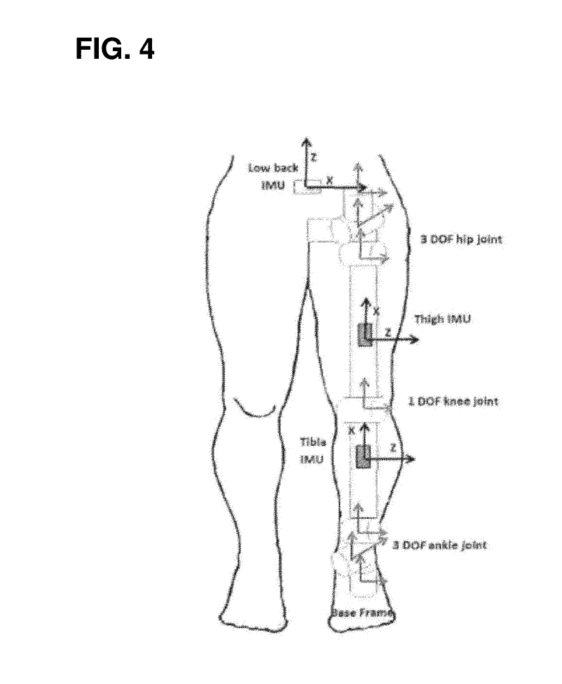

Since the IMU data is noisy and can suffer from drift, similar to [15], a kinematic model of the lower leg was applied to calculate angular velocity and linear acceleration at each time step to be used for correction of sensor estimates of these values. The kinematic model was composed of a 3 Degree of Freedom (DOF) ankle joint, 1 DOF knee joint, and 3 DOF hip joint, depicted in FIG. 4. The kinematic model predictions of the angular velocity and linear acceleration and sensor measurements of these parameters were then fused into an EKF [15], The position, velocity, and acceleration of each DOF are defined as the states to be estimated by the EKF. A constant acceleration model was used for the state propagation. See also [15].

### B. Segmentation

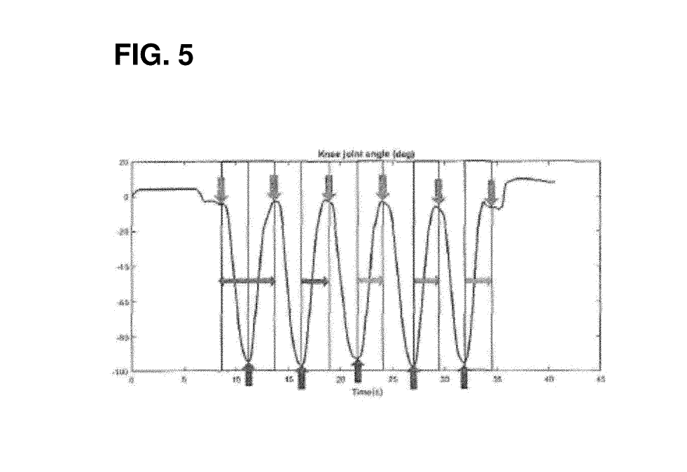

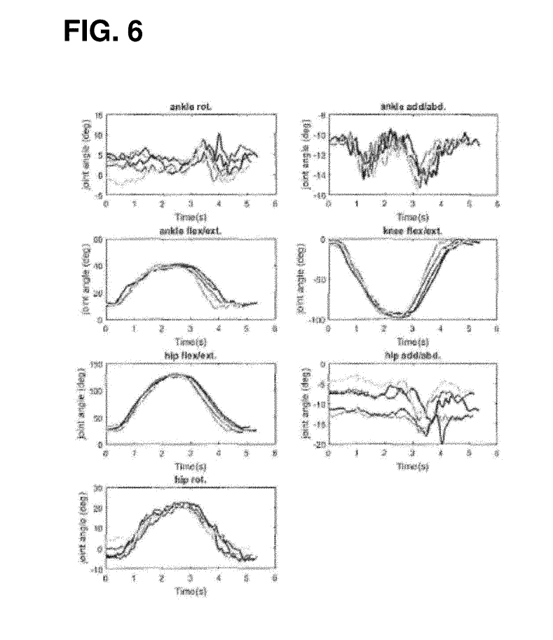

To extract a single SLS repetition from continuous time series data, the joint angle trajectory needed to be segmented before feature extraction. For segmentation, a peak detection method developed by [17] was applied to the knee flexion angle. The knee flexion was chosen for segmentation because the knee has a large ROM, and its peaks are easily detectable. A first order Butterworth filter with cutoff frequency of 0.01 rad/sample (0.3 Hz) was applied to the knee joint trajectory prior to segmentation. Note that this filter was applied only for segmentation and not for the subsequent feature extraction. The midpoints between peaks were then calculated and used as segmenting points as depicted in FIG. 5. FIG. 6 shows an example of segmented joint angles used for feature extraction (without low pass filtering).

### C. Feature Extraction

Feature extraction is necessary to transform raw time series data into relevant information about the motion to be used as predictors of DKV. Various statistical feature extraction methods have been applied for human activity recognition [18]. These methods are categorized into time domain or frequency domain methods. The most common time domain features are standard deviation (STD), mean, variance (VAR), mean absolute deviation (MAD), interquartile range (IQR), entropy, correlation between axes, and kurtosis.

Common frequency domain features include Fourier transform (FT) and discrete cosine transform (DCT).

Since in this study there would be minimal change in the frequency content of the motion, only time domain features were applied including the root mean square (RMS), STD, VAR, mean, MAD, skewness, kurtosis, range, minimum, and maximum of the joint angle, velocity and acceleration of each DOF for each segment of the data. Therefore, for each repetition of the squat, a feature vector of 210 different features was extracted.

### D. Feature Selection

Some defined features better predict DKV than others. Moreover, some features might be redundant or irrelevant, which may degrade the classification results. Selecting the most appropriate features not only helps with dimensionality reduction but also suggests the best predictors of DKV to clinicians.

A large number of feature selection techniques are available in the literature, usually categorized as filter, wrapper or embedded techniques [19]. Filter techniques select relevant features based on statistical tests. Wrapper techniques use the performance of a predefined learning algorithm as the selection criterion. In embedded techniques, feature selection occurs in parallel to model learning, so that feature selection is embedded within a classification model [19].

For this study, applied 18 different feature selection techniques from all three categories were applied. Matlab packages available from the Arizona State University [19] repository and from Pohjalainen et al. [20] were used for implementation. Wrapper methods included Random Subset Feature Selection, Sequential Forward Selection, and Sequential Floating Forward Selection.

Filter methods were Mutual Information, Statistical Dependency, Correlation based Feature Selection, ChiSqaure, Fast Correlation-Based Filter, Fisher Score, Gini Index, Information. Gain, Kruskal-Wallis, Minimum-Redundancy-Maximum-Relevance selection, Relief-Feature selection strategy, and T-test.

From embedded techniques. Sparse Multinomial Logistic Regression via Bayesian LI Regularization, Bayesian logistic regression, and Least Absolute Shrinkage and Selection Operator (LASSO) were utilized. Features selected as top ten by at least 9 methods are reported as top features. In addition to subset feature selection, feature extraction using SPCA was also applied. Matlab code developed by Barshan et al. [21] was used for SPCA implementation.

### E. Classification

For classification purposes, six different methods were applied: Support Vector Machine (SVM), Linear Multinomial Logistic Regression (LMLK), Decision Tree (DT), Naive Bayes (MB), K Nearest Neighborhood (KNN), and Random Forests. All classification techniques were implemented using Matlab 2016a. The results showed that SVM, KNN, and NB always outperformed other classifiers for this dataset. Therefore, classification results are reported for these three classifiers only.

### Example 4: Large Study—Automatic Assessment of Squat Quality and Risk of Knee Injury in the Single Leg Squat

### I. Experiments

A number (14) participants including 7 males and 7 females with mean age of 30.8±5.5, mean height of 173.8±12 Cm, and mean weight of 70.4±10.4 Kg participated in the study. For two participants, the dominant leg was the left; the other participants were right legged. Inclusion criteria were not having an active injury during the test. Both legs of one subject and the right leg of another subject had an active injury during the collection, the corresponding samples were removed from training and cross validation. Data collection was done by a clinical collaborator, ethics approval from Institutional Review Board Services was obtained prior to data collection. All participants signed a consent form prior to the start of data collection.

### A. Data Collection

Three Yost [22] IMUs were attached to the participants' low back at the level of the first sacral vertebra, the anterior thigh 10 cm above the patella aligned with the sagittal plane, and the flat surface of the shank at the level of the tibial tubercle using hypoalergic tape. Sensor placement locations are depicted in FIG. 2 Data was communicated to a nearby computer via Bluetooth communication with an average sampling rate of 90±10 Hz. Data were interpolated and resampled to the same rate of 200 Hz before subsequent analysis.

Participants were asked to perform five continuous cycles of SLS with bare feet with their toes pointing forward while keeping their weight centered over the ball of the foot and arms crossed in front of the body. They were asked to perform SLS with both the right and left legs. In case they lost balance, their legs contacted each other, or the non-weight bearing leg touched the ground, the trial was deemed unsuccessful and all cycles were repeated. Sensor placement during SLS data collection is shown in FIG. 2.

### B. Data Labelling

The participants' performance was videotaped during the tests. Videos were then reviewed by three expert clinicians trained in sports science, with an average of 9 years clinical experience. Raters were asked to label each squat repetition. The clinical rating criteria were adapted and modified from [23] and included 2 items: “Knee Valgus” and “Raters Subjective Overall Knee Injury Risk”. Each item was comprised of a three-level rating scale of 0, 1 or 2, For the knee valgus criterion, a score of 0 means no valgus, 1 means moderate knee valgus and 2 means severe knee valgus. For the overall knee injury risk criterion, a score of 0 means the individual is at no risk and no intervention is required, a score of 1 means there is mild/low risk and moderate intervention is required, and a score of 2 means the individual is at high risk and significant intervention is required. The overall knee injury risk assessment is done based on not only knee position but also trunk alignment, and pelvic and thigh motion [23].

The 14 participants performed 5 SLS repetitions with both left and right legs resulting in 140 squat repetitions to be labeled. Three categories were found in the labeled samples: samples which were unanimous (U) among raters, samples with a split (S) decision among raters, where two raters gave the same score and one gave a different score, and samples for which there was no consensus among raters, where each rater gave a different score. Labeled data statistics for each of the two criteria are summarized in Table I.

For split decision ratings, a final label based on majority vote was given to the samples. For feature selection, 4 different datasets were generated: two with combinations of both unanimous and split decision samples (for the two different criteria), and the others with only unanimous samples (again for the two criteria).

TABLE I
LABELED DATA INFORMATION

| | | | Labeled with overall | |
|---|---|---|---|---|
| U: unanimous | Labeled with knee | | risk of knee injury | |
| S: split decision | valgus criterion | | criterion | |
| H: healthy | Male # | Female # | Male # | Female # |
| Good (U, H) | 7 | 5 | 1 | 5 |
| Good (S, H) | 11 | 16 | 7 | 8 |
| Moderate (U, H) | 10 | 5 | 9 | 1 |
| Moderate (S, H) | 18 | 16 | 18 | 15 |
| Poor (U, H) | 6 | 4 | 5 | 14 |
| Poor (S, H) | 11 | 10 | 22 | 12 |
| No consensus (H) | 2 | 4 | 3 | 5 |
| Unhealthy | 5 | 10 | 5 | 10 |
| Total | 70 | 70 | 70 | 70 |

Unhealthy samples came from participants who had an active injury during the test.

For classification, only the datasets which included both split decision and unanimous samples were utilized. No consensus and unhealthy data were removed from analysis. Details of the training datasets are summarized in Table II. Additional training datasets were also generated by removing moderate exemplars for implementing 2 class classification (good vs. bad only).

TABLE II
TRAINING DATASET DETAILS

| | | | Labeled with |
|---|---|---|---|
| | | Labeled with knee | overall risk of knee |
| | | valgus criterion | injury criterion |
| Training and | Healthy - | 119 exemplars | 117 exemplars |
| validation | Unanimous | (39 good, 49 | (21 good, 43 |
| sets | or Split | moderate, 31 | moderate, 53 poor) |
| | | poor) | |
| | Healthy - | 37 exemplars | 35 exemplars |
| | Unanimous | (12 good, 15 | (6 good, 10 |
| | | moderate, 10 | moderate, 19 poor) |
| | | poor) | |
| Removed | Unhealthy | 21 exemplars | 23 exemplars |
| samples | and no- | | |
| | consensus | | |

### C. Inter and Intra-Rater Reliability (IRR)

Since there were three raters in this study, the degree of agreement (inter rater reliability), as well as consistency of the ratings by each of the raters (intra rater reliability) had to be assessed.

IRR assessment was done using the two-way mixed, consistency, average-measures ICC test [24]. Calculations were done using the irr package in R. The resulting ICC value is 0.80 for the knee valgus criterion and 0.84 for the risk of injury criterion. This indicates excellent agreement between raters according to CiCChetti guidelines [25]. To assess intra-rater reliability, 15 squat samples were randomly selected and duplicated in the dataset provided to the raters for labeling. The two-way mixed, consistency, average-measures ICC test was applied to two ratings provided for the original and duplicated samples by each rater. Intra-rater reliability results for the three raters were 1, 0.96, and 0.88 suggesting excellent reliability for all raters. IRR assessment results suggest that introduced measurement error by individual raters is minimal and SLS ratings are suitable for the purpose of classification.

### II. Results

Tables III to VI show the feature selection results for the four datasets and two different classification problems (2 classes versus 3 classes).

TABLE III
FEATURE SELECTION RESULTS FOR 2-CLASS PROBLEM
AND KNEE VALGUS CRITERION
Knee Valgus criterion-2class (good vs poor)

| | | Healthy (Unanimous + | |
|---|---|---|---|
| Healthy (Unanimous) | Nr | Split) | Nr |
| RMS of ankle IR angle | 12 | Mean of hip Flex. angle | 10 |
| | | RMS of hip Flex. angle | |
| Mean of ankle IR angle | 9 | Max of hip Flex. angle | 9 |

Nr: Number of times ranked as top ten features

TABLE IV
FEATURE SELECTION RESULTS FOR 2-CLASS
PROBLEM AND INJURY RISK CRITERION
Injury Risk criterion- 2class (good vs poor)

| | | Healthy (Unanimous + | |
|---|---|---|---|
| Healthy (Unanimous) | Nr | Split) | Nr |
| Mean of hip Flex. angle | 7 | Mean of hip Flex. angle | 14 |
| RMS of ankle IR angle | 6 | Mean of knee Flex. angle | 11 |
| RMS of ankle IR angle acceleration | | RMS of ankle IR angle | 10 |
| RMS of hip IR angle | | Max of hip Flex. angle | 9 |

Nr: Number of times ranked as top ten features

TABLE V
FEATURE SELECTION RESULTS FOR 3-CLASS
PROBLEM AND KNEE VALGUS CRITERION
Knee Valgus criterion- 3class (good vs moderate vs poor)

| | | Healthy (Unanimous + | |
|---|---|---|---|
| Healthy (Unanimous) | Nr | Split) | Nr |
| Kurtosis of angle Add. angle | 12 | Max of hip IR angle | 15 |
| Mean of ankle IR. angle | 7 | Mean of hip Flex. angle | 13 |
| RMS of angle Add. velocity | | Min of knee Flex. angle | 11 |
| Min of angle IR. velocity | | Range of hip Flex. angle | 9 |
| Max of angle IR. velocity | | RMS of hip Flex. angle | |
| STD of angle IR. acceleration | | | |

Nr: Number of times ranked as top ten features

TABLE VI
FEATURE SELECTION RESULTS FOR 3-CLASS
PROBLEM AND INJURY RISK CRITERION
Injury Risk criterion- 3 class (good vs moderate vs poor)

| | | Healthy (Unanimous + | |
|---|---|---|---|
| Healthy (Unanimous) | Nr | Split) | Nr |
| STD of hip IR velocity | 11 | Max hip Flex. angle | 10 |
| Range of hip Flex. angle | 10 | Mean of hip Flex. angle | 9 |
| MAD of hip IR velocity | | VAR of ankle Add. velocity | |
| VAR of hip IR velocity | 9 | | |
| RMS of hip IR angle | | | |

Nr: Number of times ranked as top ten features

The feature selection results for the unanimous data in both 2 class and 3 class problems reveal that ankle internal rotation/adduction features are the most important predictors of DKV, while in terms of risk of injury, hip internal rotation/flexion and ankle internal rotation features are more discriminative. Another observation from the unanimous data is that in the 2 class problem, joint angle features appear as predictors, while for the 3 class problem, joint velocity plays a significant role. These results are in agreement with the pilot study (14).

On the other hand, when the data include both unanimous and split decision samples, flexion angles, particularly hip flexion, frequently appear as predictors of the knee valgus or risk of injury. Analyzing the flexion joint angles of the SLS repetitions revealed that those labeled as good tend to have increased torso bending during the motion, probably to maintain better balance and have more control over the motion.

The dimensionality of the training data was reduced by keeping only the identified important features. Classification techniques are applied to the reduced dimensionality dataset including both unanimous and split decision data, using the labels from the two criteria. Results for both 10 fold and LOSO cross-validations are reported in Tables VII to X. SPCA dimensionality reduction method results are also provided.

TABLE VII
CLASSIFICATION RESULTS FOR 2-CLASS
PROBLEM AND KNEE VALGUS CRITERION
2 class problem accuracy (%)
Knee Valgus Criterion

| | 10F-CV | | LOSO-CV | |
|---|---|---|---|---|
| Dim. Red. | Subset of | | Subset of | |
| Method | selected features | SPCA | selected features | SPCA |
| SVM | 92.71 | 93.14 | 87.5 | 89.77 |
| NB | 92.71 | 90.42 | 87.5 | 84.1 |
| KNN | 92.85 | 92.57 | 86.36 | 86.36 |

TABLE VIII
CLASSIFICATION RESULTS FOR 3-CLASS
PROBLEM AND KNEE VALGUS CRITERION
3 class problem accuracy (%)
Knee Valgus Criterion

| | 10F-CV | | LOSO-CV | |
|---|---|---|---|---|
| Dim. Red. | Subset of | | Subset of | |
| Method | selected features | SPCA | selected features | SPCA |
| SVM | 66.87 | 73.5 | 65.4 | 67.4 |
| NB | 60.99 | 67.7 | 57 | 59.6 |
| KNN | 70.12 | 72.15 | 67.6 | 67.2 |

TABLE IX
CLASSIFICATION RESULTS FOR 2-CLASS
PROBLEM AND RISK OF INJURY CRITERION
2 class problem accuracy (%)
Risk of Injury Criterion

| | 10F-CV | | LOSO-CV | |
|---|---|---|---|---|
| Dim. Red. | Subset of | | Subset of | |
| Method | selected features | SPCA | selected features | SPCA |
| SVM | 92.11 | 86.9 | 77.46 | 84.1 |
| NB | 95.25 | 85.39 | 87.39 | 82.25 |
| KNN | 93.7 | 87.32 | 85.87 | 79.35 |

TABLE X
CLASSIFICATION RESULTS FOR 3-CLASS
PROBLEM AND RISK OF INJURY CRITERION
3 class problem accuracy (%)
Risk of Injury Criterion

| | 10F-CV | | LOSO-CV | |
|---|---|---|---|---|
| Dim. Red. | Subset of | | Subset of | |
| Method | selected features | SPCA | selected features | SPCA |
| SVM | 67.17 | 74.4 | 61.67 | 74.87 |
| NB | 66.34 | 68.67 | 66.67 | 61.27 |
| KNN | 67.86 | 76.3 | 66.26 | 73.27 |

Classification results for 10F-CV showed that distinguishing between good and poor squats is achievable with a promising accuracy (93%). For the three class problem, however, the best achieved accuracy was 74%. LOSO-CV results were slightly lower, with best accuracy of 90% for 2 class and 68% for the 3 class problems. With respect to predicting the risk of injury, the best achieved accuracy using 10F-CV was 95% for the 2 class and 76% for the three class problem. Using the LOSO-CV, the best accuracy for 2 class was 87% and for 3 class problem was 75%.

### 1. Ankle Only Features

In the pilot data analysis [14], the ankle IR features were found to be the best predictors of the DKV, which suggest that it is possible to use only one sensor on the tibia (saving time and simplifying the test protocol) and still have good classification accuracy. To confirm this hypothesis with the larger datasets, we used feature selection on only ankle extracted features (90 out of 210 features) and found that ankle IR velocity, angle and acceleration features are the best predictors in the absence of hip or knee information. The classification was also repeated using ankle only features. The best achieved results using ankle only features and the percentage of change in accuracy in comparison to the best reported results using all joints' features are shown in Tables XI and XII.

The results from Tables XI and XII indicate that there is less than 4% drop in accuracy for risk of injury detection using only ankle information (one tibia sensor) and less than 9% drop for knee valgus detection, suggesting that one sensor can be used to simplify the data collection procedure.

TABLE XI
BEST ACHIEVED CLASSIFICATION RESULTS
FOR 10F-CV USING ANKLE FEATURES

| 10F- CV accuracy (%) | |
|---|---|
| Knee Valgus Criterion | Risk of Injury Criterion |

| Best | ankle only | change in | ankle only | change in |
|---|---|---|---|---|
| results | features | accuracy | features | accuracy |
| 2 class | 84.14 | −9% | 91.67 | −3.6% |
| 3 class | 67.5 | −6% | 77.13 | +1.23% |

TABLE XII
BEST ACHIEVED CLASSIFICATION RESULTS
FOR LOSO-CV USING ANKLE FEATURES

| LOSO- CV accuracy (%) | |
|---|---|
| Knee Valgus Criterion | Risk of Injury Criterion |

| Best | ankle only | change in | ankle only | change in |
|---|---|---|---|---|
| results | features | accuracy | features | accuracy |
| 2 class | 85.23 | −4.54% | 83.7 | −3.69% |
| 3 class | 60.4 | −7.2% | 73.5 | −1.37% |

### 2. Gender Specific Analysis

It was hypothesized that men and women might have different biomechanical characteristics and movement strategies which result in different predictors. To test this hypothesis, two different datasets were made including women only (60 samples) and men only (65 samples) healthy data. Feature selection methods were applied to both datasets separately. The results reported in Tables XIII, XIV, XV, XVI showed that different features were selected when the data is segregated by sex. For the male dataset, the features selected were the hip and knee flexion features. For females, hip and ankle IR features were selected. Based on this finding, it was also tested whether men-specific and women-specific classifiers might work better than a general classifier for both genders. The SVM classifier was used for the two data set and results are compared to general classifier results (developed in previous section) in Tables XVII, XVIII.

Classification results show that for women, in all cases, the women-specific classifier works better than the general classifier. For men, the same holds for risk of injury index. The only noticeable exception is the 2-class classification with knee valgus criterion, for which the general classifier is better.

TABLE XIII
GENDER SPECIFIC FEATURE SELECTION RESULTS FOR
2-CLASS PROBLEM AND KNEE VALGUS CRITERION
Knee Valgus - 2class (good vs poor)

| Males | Nr | Females | Nr |
|---|---|---|---|
| mean of knee Flex. angel | 13 | rms of ank IR velocity | 10 |
| max of hip Flex. velocity | 11 | std of ank IR. velocity | 7 |
| rms of hip Flex. angle | 7 | rms ank IR acceleration | 6 |
| std of hip Flex. angle | | mad ank IR aceleration | |
| mad of hip Flex. angle | | var of ank IR. velocity | |
| | | mad of ank IR. velocity | |
| | | mean ank Add. velocity | |

Nr: Number of times ranked as top ten features

TABLE XIV
GENDER SPECIFIC FEATURE SELECTION RESULTS FOR
2-CLASS PROBLEM AND INJURY RISK CRITERION
Injury Risk - 2class (good vs poor)

| Males | Nr | Females | Nr |
|---|---|---|---|
| mad of hip Flex. velocity | 10 | std of hip IR velocity | 11 |
| kurtosis of hip Flex. velocity | 8 | mean of knee Flex angle | 9 |
| rms of hip Flex. velocity | 7 | mean of hip Flex angle | 8 |
| | | var of hip IR velocity | |

Nr: Number of times ranked as top ten features

TABLE XV
GENDER SPECIFIC FEATURE SELECTION RESULTS FOR
3-CLASS PROBLEM AND KNEE VALGUS CRITERION
Knee Valgus - 3class (good vs poor vs moderate)

| Males | Nr | Females | Nr |
|---|---|---|---|
| rms of hip Flex. angle | 13 | rms of hip Add. velocity | 8 |
| max of hip Flex. velocity | 12 | mad of hip IR. acceleration | 7 |
| mean of hip Flex. velocity | 9 | var of ank IR. velocity | 6 |
| std of hip Flex. angle | 8 | mad ank IR. velocity | |
| var of hip Flex. angle | | std of hip Add. velocity | |
| mad of hip Flex. angle | | | |
| mean of hip Flex. angle | | | |

Nr: Number of times ranked as top ten features

TABLE XVI
GENDER SPECIFIC FEATURE SELECTION RESULTS FOR
3-CLASS PROBLEM AND INJURY RISK CRITERION
Injury Risk - 3class (good vs poor vs moderate)

| Males | Nr | Females | Nr |
|---|---|---|---|
| std of hip Flex. velocity | 9 | std of hip IR. velocity | 9 |
| | | mad ank IR. acceleration | |
| mean of hip Flex. velocity | 8 | std of ank IR. velocity | 8 |
| kurtosis of knee Flex. acceleration | | rms of ank IR. acceleration | |
| kurtosis knee Flex. velocity | 7 | | |

Nr: Number of times ranked as top ten features

TABLE XVII
GENDER SPECIFIC CLASSIFICATION
RESULTS OF FOR 10F-CV

| | SVM-10F-CV accuracy % | |
|---|---|---|
| Classifier | 2 Class | 3 Class |

| type | Valgus | Risk | Valgus | Risk |
|---|---|---|---|---|
| Men only | 90.5 | 97.3 | 84 | 73.6 |
| Women only | 94.3 | 99.8 | 74.7 | 84.9 |
| General- | 93.1 | 95.3 | 73.5 | 74.4 |
| best results | | | | |

TABLE XVIII
GENDER SPECIFIC CLASSIFICATION
RESULTS OF FOR LOSO-CV

| | SVM-LOSO-CV accuracy % | |
|---|---|---|
| Classifier | 2 Class | 2 Class |

| type | Valgus | Risk | Valgus | Risk |
|---|---|---|---|---|
| Men only | 72.7 | 91.7 | 82.7 | 80 |
| Women only | 93.2 | 100 | 74.6 | 86.8 |
| General- | 89.8 | 87.4 | 67.6 | 74.9 |
| best results | | | | |

### III. Discussion and Conclusions

In this study, an automatic assessment method was developed to evaluate single leg squat quality. Two criteria were used for labeling: amount of inward knee movement during the task (knee valgus), and holistic risk assessment by expert clinician raters. SLS data of 14 volunteers were collected and two data sets were generated: one included the data with unanimous agreement among raters and the other dataset was a combination of full and partial agreement of labeled data. 18 feature selection methods were applied to the datasets to find the best predictors of knee valgus and risk of knee injury. The feature selection results for only unanimous data suggested ankle IR/Add and hip IR/flex features to be correlated with DKV and risk of injury, respectively. However, for both unanimous and split decision data, hip/knee flexion angle features were highlighted as the predictors of both DKV and risk of injury.

The participants were not instructed to keep their torso upright during the data collection. The fact that hip flexion angle features appeared as best predictors of DKV and risk of injury in the full dataset indicates that other motion behaviors are also associated with knee valgus or risk, and that different test protocols and instructions can lead to different results. Changing test instructions can change feature selection results, which has to be considered in the clinical application of the developed tool.

Three common classification techniques were applied to the datasets. The LOSO-CV results suggest that discriminating of poor squats from good ones is achievable with promising accuracy of 90%. Changing the problem to multiclass (adding moderate squats) drops the accuracy by 22%. Screening participants at high risk of injury from those at no risk can be done by 87% accuracy and adding mild risk subjects drops accuracy by 12%.

The achieved performance in the 2 class problem is comparable to Whelan et al [13]. It was also shown that the classification generalizes to unseen participants and investigate 3 class classification. Unlike Whelan et al., features over joint angles, velocities and accelerations are used, which are clinically interpretable parameters.

The results of gender specific classifiers suggest that developing separate classifiers for men and women improves classification results significantly and strengthen our hypothesis about different biomechanical characteristics or movement strategies in men and women, which worth further analysis in future studies.

### REFERENCES

[1] P. Hattam and A. Smeatham, Special tests in musculoskeletal examination: an evidence-based guide for clinicians. Elsevier Health Sciences.
[2] D. A. Padua. D. R. Bell, and M. A. Clark, “Neuromuscular characteristics of individuals displaying excessive medial knee displacement,” J. Athl. Train., vol. 47, no. 5, pp. 525-536, 2012.
[3] A. L. Gornitzky. A. Loll. J. L. Yellin, P. D. Fabricant, J. T. Lawrence, and T. J. Ganley, “Sport-Specific Yearly Risk and Incidence of Anterior Cruciate Ligament Tears in High School Athletes: A Systematic Review and Meta-analysis,” Am. J. Sports Med., p. 0363546515617742-, 2015.
[4] M. V. Patemo, “Incidence and predictors of second anterior cruciate ligament injury after primary reconstruction and return to sport,” J. Athl. Train., vol. 50, no. 10, pp. 1097-1099, 2015.
[5] H. J. Silvers and B. R. Mandelbaum, “Prevention of anterior cruciate ligament injury in the female athlete,” Br. J. Sports Med., vol. 41, no. Suppl 1, pp. i52-i59, 2007.
[6] M. Walden, M. Hagglund, H. Magnusson, and J. Ekstrand, “ACL injuries in men's professional football: a 15-year prospective study on time trends and return-to-play rates reveals only 65% of players still play at the top level 3 years after ACL rupture,” Br. J. Sports Med., vol. 50, pp. 744-750, 2016.
[7] L. Herrington, “Knee valgus angle dming single leg squat and landing in patellofemoral pain patients and controls,” Knee, vol. 21, no. 2, pp. 514-517, 2014.
[8] B. L. Zeller. J. L. McCrory, W. Ben Kibler, and T. L. Uhl, “Differences in kinematics and electromyographic activity between men and women during the single-legged squat.,” Am. J. Sports Med., vol. 31, no. 3, pp. 449-56, 2003.
[9] N. F. N. Bittencourt, J. M. Ocarina, L. D. M. Mendonca, T. E. Hewett, and S. T. Fonseca, “Foot and hip contributions to high frontal plane knee projection angle in athletes: a classification and regression tree approach.,” J. Orthop. Sports Phys. Ther., vol. 42, no 12, pp. 996-1004, 2012.
[10] M. R. Stiffler, A. P. Pennuto, M. D. Smith. M. E. Olson, and D. R. Bell, “Range of Motion, Postural Alignment, and LESS Score Differences of Those With and Without Excessive Medial Knee Displacement,” Clin. J. Sport Med., vol. 25, no. I, pp. 61-66, 2014.
[11] V. Ugalde, C. Brockman, Z. Bailowitz, and C. D. Pollard, “Single Leg Squat Test and Its Relationship to Dynamic Knee Valgus and Injury Risk Screening,” Phys. Med. Rehabil., vol. 7, no. 3, pp. 229-235, 2015.
[12] D. A. Padua. S. W. Marshall, M. C. Boling. C. A. Thigpen, W. E. Garrett, and I. Anthony. “The Landing Error Scoring System (LESS) Is a Valid and Reliable Clinical Assessment Tool of Jump-Landing Biomechanics: The JUMP-ACL Study.” Am. J. Sports Med., vol. 37, pp. 1996˜2002, 2009.
[13] D. Whelan. M. 0. Reilly, T. Ward, and B. Caulfield, “Evaluating Performance of the Single Leg Squat Exercise with a Single Inertial Measurement Unit,” in Workshop on ICTs for improving Patients Rehabilitation Research Techniques, 2015, pp. 144˜147.
[14] R. Kianifar, A. Lee. S. Raina, and D. Kulic, “Classification of Quality of Squats with Inertial Measurement Units in the Single Leg Squat Mobility Test,” in IEEE Engineering in Medicine and Biology Society, 2016.
[15] J. F. S. Lin and D. Kulic, “Human pose recovery using wireless inertial measurement units,” Physiol. Meas., val. 33, no. 12, pp. 2099˜115, 2012.
[16] T. Seel, J. Raisch, and T. Schauer, “IMU-based joint angle measurement for gait analysis,” Sensors, val. 14, no. 4, pp. 6891˜909, 2014.
[17] T. O. Haver, “A Pragmatic Introduction to Signal Processing.” [Online]. Available: http://terpconnect.umd.edu/-toh/spectrum/PeakFindingandMeasurement.htm.
[18] O. D. Lara and M. A. Labrador. “A Survey on Human Activity Recognition using Wearable Sensors.” Commun. Surv. Tutorials, vol. 15, no. 3, pp. 1192-1209, 2013.
[19] Z. Zhao, F. Morstatter, S. Sharma. S. Alelyani A. Anand, and H. Liu, “Advancing Feature Selection Research,” ASU Featur. Sel. Repos. Arizona State Univ., pp. 1˜28, 2010.
[20] J. Pohjalainen, O. Rasanen, and S. Kadioglu. “Feature selection methods and their combinations in high-dimensional classification of speaker likability, intelligibility and personality traits.” Comput. Speech Lang., vol. 29, no. I, pp. 145˜171, 2015.
[21] E. Barshan, A. Ghodsi, Z. Azimifar, and M. Zolghadri Jahromi, “Supervised principal component analysis: Visualization, classification and regression on subspaces and submanifolds,” Pattern Recognit., vol. 44, no. 7, pp. 1357˜1371, 2011.
[22] “YOST.” [Online]. Available: https://yostlabs.com/.
[23] L. Herrington and A. Munro, “A Preliminary Investigation to Establish the Criterion Validity of a Qualitative Scoring System of Limb Alignment during Single Leg Squat and Landing,” J Exerc Sport. Orthop., val. I, no. 2, pp. 1˜6, 2014.
[24] K. A. Hallgren, “Computing Inter-Rater Reliability for Observational Data: An Overview and Tutorial,” Tutor Quant Methods Psychol, vol. 8, no. I, pp. 23˜34, 2012.
[25] D. V Cicchetti, “Guidelines, criteria, and rules of thumb for evaluating normed and standardized assessment instruments in psychology,” Psychol. Assess., vol. 6, no. 4, pp. 284˜290, 1994.
[26] R. Bailey, J. Selfe, and J. Richards, “The single leg squat test in the assessment of musculoskeletal function: a review,” Physiother. Pract. Res., vol. 32, no. 2, pp. 18-23, 2011.
[27] S. J. Preece, J. Y. Goulermas, L. P. Kenney et al., “Activity identification using body-mounted sensorsa review of classification techniques,” Physiol. Meas., vol. 30, no. 4, p. R1, 2009.
[28] A. Almangoush, L. Herrington, and R. Jones, “A preliminary reliability study of a qualitative scoring system of limb alignment during single leg squat,” Physical Therapy and Rehabilitation, vol. 1, no. 1, p. 2, 2014.
[29] J. F.-S. Lin, M. Karg, and D. Kulic, “Movement primitive segmentation for human motion modeling: A framework for analysis,” IEEE Trans. Hum.-Mach. Syst., 2015.

1. A method of assessing the risk of knee injury in a subject, comprising:
determining a performance parameter of the squatting leg of the subject performing a leg squat, wherein the parameter is measured by means of one or more sensor(s) placed on the subject,
analyzing the parameter to obtain an indication of the risk of knee injury in the subject.

2. The method of claim 1, wherein the leg squat is a single leg squat (SLS).

3. The method of claim 2, wherein the sensor is an inertial measurement unit (IMU) composed of a set of three accelerometers measuring linear accelerations, and three gyroscopes measuring angular velocities and a magnetometer measuring earth's magnetic field.

4. The method of claim 3, wherein several parameters are measured by the sensor including joint angle, velocity and acceleration of the squatting leg.

5. The method of claim 4, wherein a set of three IMU's are employed.

6. The method of claim 5, wherein the IMU's are located individually at the lower back, at the thigh and at the tibia of the subject.

7. The method of claim 6, wherein dynamic knee valgus (DKV) is assessed by analysis of the parameters and DKV is used as an indication of the risk of the knee injury in the subject.

8. The method of claim 7, wherein readout of the sensor is received remotely.

9. The method of claim 7, wherein the analysis includes evaluation of flexion at the hip and knee and hip and ankle rotation.
